(19) United States
(12) Patent Application Publication
(10) Pub. No.: US 2019/0046670 A1
(43) Pub. Date: Feb. 14, 2019

(54) WIRELESS ELECTROSTIMULATION TO ERADICATE IMPLANTED DEVICE ASSOCIATED BIOFILMS

(71) Applicants: Dacheng Ren, Longchang (CN); Hao Wang, Nanjing (CN)

(72) Inventors: Dacheng Ren, Longchang (CN); Hao Wang, Nanjing (CN)

(73) Assignee: SYRACUSE UNIVERSITY, SYRACUSE, NY (US)

(21) Appl. No.: 16/100,302

(22) Filed: Aug. 10, 2018

Related U.S. Application Data
(60) Provisional application No. 62/543,570, filed on Aug. 10, 2017.

(51) Int. Cl. A61L 2/03; A61N 1/30; A61N 1/36; A61L 2/08

(52) U.S. Cl. CPC A61L 2/03; A61N 1/306; A61N 1/0428; A61L 2/08; A61N 1/36038

(57) ABSTRACT

A medical device having DC treatment electrodes that can be wirelessly induced to produce the appropriate electrical current across the implant to eradicate any biofilm that has formed on the implant shell, such as that of a cochlear implant. The implant may also be configured to sample any changes in the redox properties of electrodes to detect the formation of biofilm on the implant and provide a notification that DC treatment is needed.

### CROSS-REFERENCE TO RELATED APPLICATIONS

The present application claims priority to U.S. Provisional Application No. 62/543570, filed on Aug. 10, 2017.

### BACKGROUND OF THE INVENTION

### 1. Field of the Invention

The present invention relates to the treatment of biofilms forming on medical implants and, more particularly, to a system and method for treating biofilms that form on cochlear implants.

### 2. Description of the Related Art

A cochlear implant is an electronic device that is implanted in a patient with deafness to enhance hearing. A cochlear implant usually has a polysilicon housing that contains antenna coils, a receiver/stimulator modulus, a magnet, and electrode arrays that connect to the vestibulocochlear nerve system. As is the case with other implanted devices, a cochlear implant is prone to bacterial colonization and subsequent infections. The infections can lead to ulcers, or even inflammation of brain tissue, thereby requiring surgical intervention such as implant relocation, fixation, and even explanation.

Bacterial biofilms play an important role in recalcitrant cochlear implant infections. The major causative agents of cochlear implant infections include Staphylococcus aureus, Pseudomonas aeruginosa and Streptococcus species. S. aureus is a common strain isolated from lesions of cochlear implant infection and usually leads to ulcer and swelling, while P. aeruginosa is more related to chronic infections. Recently, Streptococcus species, especially S. pneumonia, have attracted attention due to an associated high risk of meningitis among children with cochlear implant implants.

Direct current (DC) when applied for relatively long periods (from several hours to days) has been shown to have bactericidal effects in treatment including biofilms. DC has also been shown to have a synergy with antibiotics in bacterial killing. However, conventional approaches for applying DC all require a direct connection between the requisite electrodes and a power source. As a result, conventional approaches for treating cochlear implants with DC would require piercing the skin of the patient with the wiring needed to provide current for treatment. Accordingly, there is a need in the art for a non-invasive approach for applying DC to cochlear implant to treat bacterial infections.

### BRIEF SUMMARY OF THE INVENTION

The present invention provides infection control for medical implants using DC, by inducing DC wirelessly using a magnetic field. Implantable biomedical devices, such as pacemakers, deep brain stimulators, and cochlear implants, are provided an inductive coil located on the internal sides of the human body, respectively, for the delivery of electrical power by electromagnetic induction. Electromagnetic inductive coupling powers the implant devices wirelessly without any piercing of the skin. The delivered electrical power is used to power a circuit that applies an effective amount of DC to the medical implant to treat biofilm infections.

Wirelessly derived DC was found to have a strong effect in killing P. aeruginosa and S. aureus planktonic and biofilm cells, and the levels of effective DC are within the range that is known to be safe to human. The present invention demonstrates that biofilm cells can be effectively killed by using electromagnetic induction to deliver DC wirelessly from a power source. After treatment with 5-50 μA/cm² of wirelessly delivered DC over 6 hours, the viability of biofilm cells was reduced by approximately 4 logs and 2 logs for P. aeruginosa and S. aureus, respectively. The coupling induction technology is applicable to implanted medical devices since it can deliver power and signals wirelessly without skin piecing wiring, and overcome the infection problems as well as lifetime limitation if a battery was used to power.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a schematic of a testing apparatus according to the present invention;

FIG. 2 is a graph of the viability of P. aeruginosa planktonic cells after treatment with 0.05 and 0.5 μA/cm² DC in 0.85% NaCl;

FIG. 3 is a graph of the viability of P. aeruginosa biofilm cells after treatment with 0.05 and 0.5 μA/cm2 DC in 0.85% NaCl;

FIG. 4 is a graph of the viability of P. aeruginosa biofilm cells after treatment with 5, 25 and 50 μA/cm² DC in 0.85% NaCl;

FIG. 5 is a graph of the viability of S. aureus biofilm cells after treatment with 5, 25 and 50 μA/cm² DC in 0.85% NaCl;

FIG. 6 is a graph of the viability of P. aeruginosa biofilm cells after treatment with Tob alone, DC alone or concurrent treatment with Tob and DC. Treatment medium: 0.85% NaCl. DC level: 5 μA/cm², Tob dosage: 4.5 μg/mL;

FIG. 7 is a schematic of a medical implant according to the present invention;

FIG. 8 is a schematic of a system for inducing DC current in medical implant according to the present invention.

### DETAILED DESCRIPTION OF THE INVENTION

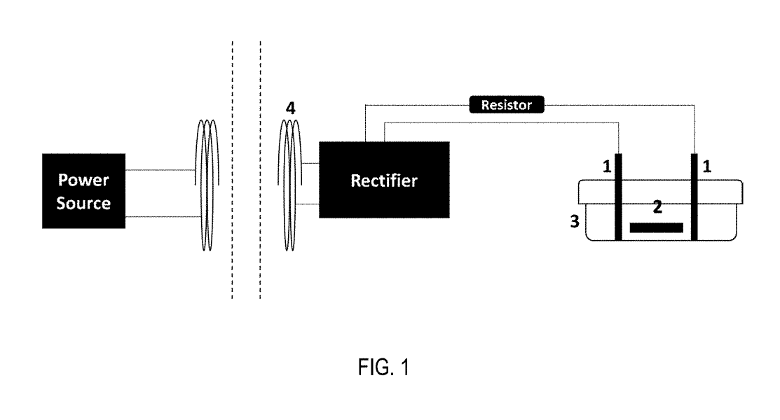

Referring to the figures, wherein like numerals refer to like parts throughout, there is seen in FIG. 1 an experimental system 1 for establishing whether induced DC in a medical implant would reduce bacterial biofilms. The treatment circuit was constructed with two electrodes 1 on the opposite sides of a 35 mm petri dish 3 (Thermo Fisher Scientific, Pittsburg, Pa., U.S.). To deliver DC wirelessly, electrodes 1 were connected to a rectifier that is connected to the copper receiver coil 4 (10 turns, diameter was 10 cm). The total current level of treatment circuit was controlled by using different resistors (10 k-10 M ohm) (Scheme 1). The receiver coil was placed in the center of a wireless charging pad as power source.

Materials and Methods

Bacteria Strains and Growth Media

P. aeruginosa PAO1 was obtained from Department of Genome Sciences, University of Washington and S. aureus ALC2085 (strain RN6390 containing pALC2084) was obtained from the Sauer lab at Binghamton University. Both strains were cultured in Luria-Bertani (LB) medium (Niepa, et al. 2012b) containing 10 g/L tryptone, 5 g/L Yeast extract and 10 g/L NaCl. Both strains were routinely cultured overnight at 37° C. with shaking at 200 rpm.

Biofilm Formation

Biofilms were formed on polydimethylsiloxane (PDMS) blocks (1 cm×0.5 cm). Briefly, 25 μL overnight culture of planktonic cells was used to inoculate a petri dish containing 25 mL of LB medium and PDMS blocks. The culture was incubated at 37° C. for 24 h without shaking. Then the PDMS blocks with biofilms were removed from the petri dish and washed gently with 0.85% NaCl solution for DC treatment.

DC Treatment of Planktonic Cells

The P. aeruginosa PAO1 or S. aureus ALC2085 overnight cultures were washed with 0.85% NaCl solution, and then resuspended in 3 ml 0.85% NaCl solution to approximately 1×10⁸ cells/ml. After being transferred to a petri dish, the cells were treated with DC for varying duration (2, 4 or 6 hours). The untreated samples were used as controls. After treatment, the number of viable cells was quantified by counting colony forming units (CFUs) in the solution.

DC Treatment of Biofilms

The DC treatment of biofilm was carried out in 3 mL 0.85% NaCl solution. A PDMS block with P. aeruginosa PAO1 or S. aureus ALC2085 biofilm was placed between two electrodes. The biofilm was treated with DC for 2, 4 or 6 h. The untreated samples were used as controls. After treatment, each PDMS block was transferred to a 10 mL tube containing 2 mL 0.85% NaCl solution. The biofilm cells were removed from the surface by gentle sonication for 1 min. The number of viable cells detached from PDMS blocks was quantified by counting CFU in the solution.

Tobramycin (Tob) also used to evaluate the synergy with low level DC. PDMS blocks with P. aeruginosa PAO1 biofilm were treated with 5 μA/cm² DC and 4.5 μg/mL Tob for 2 or 6 h. The number of viable biofilm cells was quantified by counting CFU.

Power calculation

The electric power consumption of DC treatment can be calculated using the equation below:

Power=I²×ρ×d

where I is current level; ρ is average resistivity (e. g. soft tissue between electrodes); and d is the distance between two electrodes.

Results

Effects of DC on P. aeruginosa Planktonic Cells

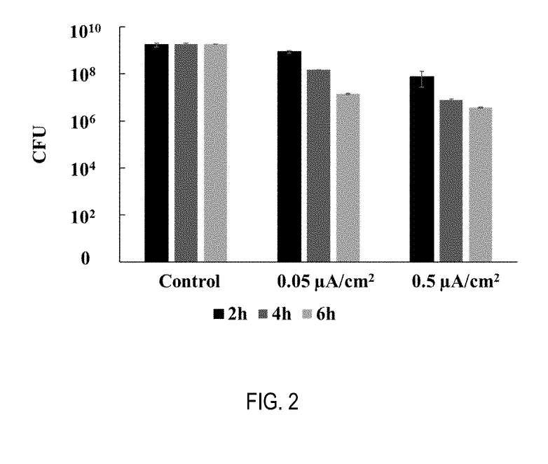

As shown in FIG. 2, the killing effects of DC on P. aeruginosa planktonic cells were time dependent. Specifically, treatment with 0.05 μA/cm² killed 0.3, 1.1 and 2.1 logs by applying this DC for 2, 4 and 6 h, respectively. When the DC level increased to 0.5 μA /cm², the killing effect was 1.3, 2.4 and 2.7 logs for 2, 4 and 6 h of treatment, respectively. These results show that killing is more significant with increases in time and current level.

Effects of DC on P. aeruginosa and S. aureus Biofilms

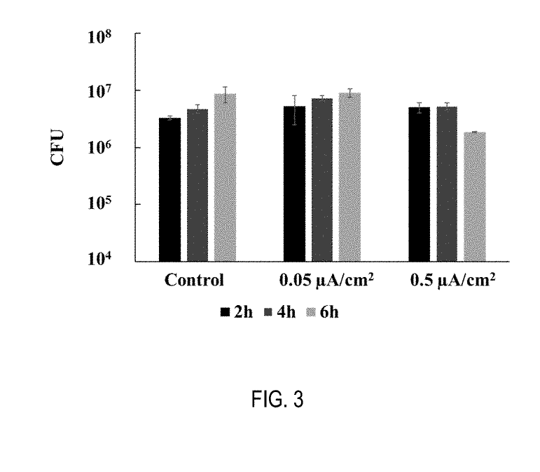

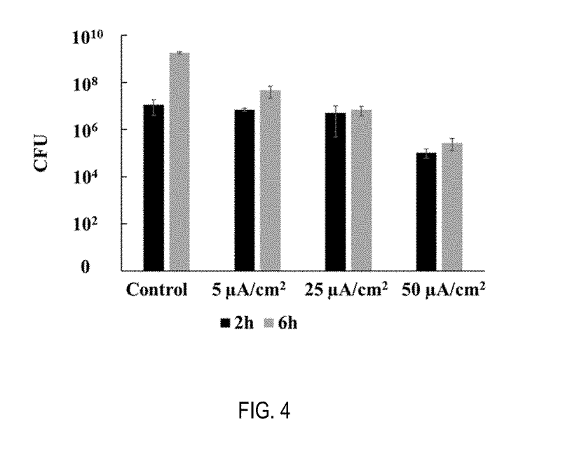

The biofilm of P. aeruginosa was more tolerant to DC treatment than its planktonic cells. The maximum killing effect (approximately 3.9 logs) on P. aeruginosa biofilms was observed under the condition of 50 μA/cm² DC for 6 h treatment, which was reduced to 2 logs when the DC treatment was shortened to 2 h (FIG. 3). At lower current levels, the 6 h treatment with 0.5, 5 or 25 μA/cm² killed 0.7, 1.6 and 2.4 logs, respectively. Thus, biofilm cells are more tolerant to DC treatment as expected (FIG. 3). No significant killing was observed at any of these current levels with the treatment time shortened to 2 h (FIGS. 3 and 4).

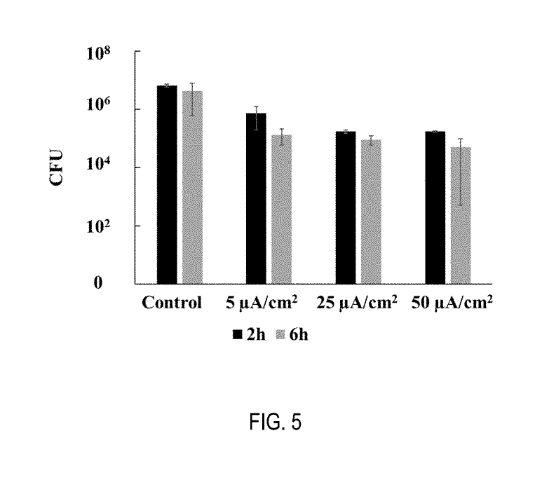

Similar killing effects were also observed for S. aureus biofilms under the same treatment conditions. For example, the number of viable S. aureus biofilm cells was reduced by 1.6 and 2 logs after treatment with 50 μA/cm² DC for 2 and 6 h, respectively. The 25 μA/cm² DC showed similar killing activities on S. aureus biofilms (1.6 and 1.7 log for 2 and 6 h treatment, respectively); and 5 μA/cm² DC only exhibited significant killing effect (1.5 log) with 6 h treatment (FIG. 5).

Synergy Between DC and Antibiotics in Killing P. aeruginosa Biofilms

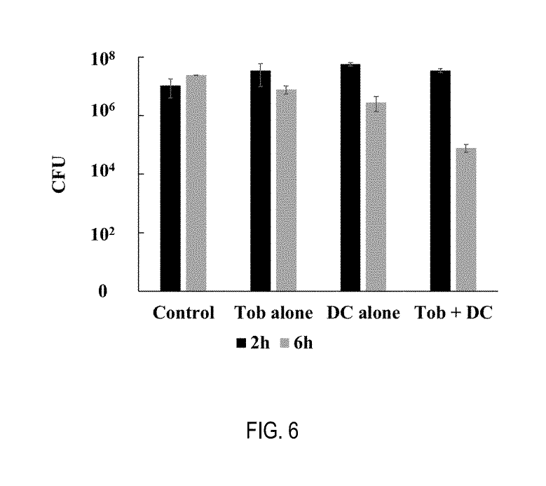

When P. aeruginosa biofilm was treated with DC and tobramycin (Tob) for 6 h, the maximum killing effect (2.5 logs) was observed under the condition of 5 μA DC/cm² and 4.5 μg/mL tobramycin. In comparison, treatment with 5 μA/cm² DC or 4.5 μg/mL Tob alone only showed 0.5 log and 0.9 log of killing, respectively (FIG. 6). There was no significant killing for 2 h treatment with the same level of DC and tobramycin.

Power Consumption of DC Treatment System

The power consumption for DC treatment in our system in vivo should be 0.15 (0.5 μA/cm² DC) to 1.5 mW (50 μA/cm² DC) by calculation.

Discussion

Different levels of direct currents (DC) and alternative currents (AC) have been demonstrated to kill biofilm cells in the presence or absence of antibiotics (Brinkman, et al. 2016; del Pozo, et al. 2009; Schmidt-Malan, et al. 2015; Voegele, et al. 2015). Our group recently found synergetic effects between low level DC and the antibiotic tobramycin in killing P. aeruginosa biofilm and persister cells (Niepa, et al. 2012a ; Niepa, et al. 2012b ; Niepa, et al. 2016). However, all those systems require physical connection between the treatment component and power source. Such setup requires skin piecing or battery if applied to implanted medical devices, which would bring problems such as limited device lifetime, pain associated with treatment, and risk of additional infection. In this study, we delivered the electric power wirelessly by induction coupling and achieved effective killing of bacteria including biofilm cells and synergy with antibiotics. Our results show that the viability of P. aeruginosa and S. aureus biofilm cells can be reduced by up to 4 logs with 6 h treatment of 5-50 μA/cm² DC. The lower level DC (0.05-0.5 μA/cm²) also showed the killing effect on planktonic cells, but not biofilms. Synergetic effect was observed for concurrent treatment with 5 μA/cm² DC and 4.5 μg/ml tobramycin for 6 h.

Typical power supply for cochlear implants is approximately 10 mW (Wei and Liu 2008), which is 7-70 folds higher than the power requirement of DC treatment in vivo based on our calculation. Hence, it is possible to take the original electronic units of cochlear implant and rewire the system to provide both hearing aid and infection control. Formation of biofilms can be monitored by measuring impedance and the therapeutic DC can be delivered by adding additional control modules. The maximum current density of DC that can be safely applied in human brain is 2 mA/cm² (Murray, et al. 2015; Valic, et al. 2009; Neuroelectric's). The DC density used in this design (approximately 5-50 μA/cm²) is much lower than that and thus is expected to be safe. The published long-term DC current level of cochlear implant is approximately 0.3 μA (Clark 2006). In our system, we can reduce the viability of P. aeruginosa planktonic cells by 2 logs with only 0.05 μA/cm² DC in 6 h, although the biofilm cells may require longer treatment time or higher DC level (still in the safe range). Based on our results, we speculated that biofilm killing can be obtained by nano-amp level DC with longer treatment, e.g. >24 h.

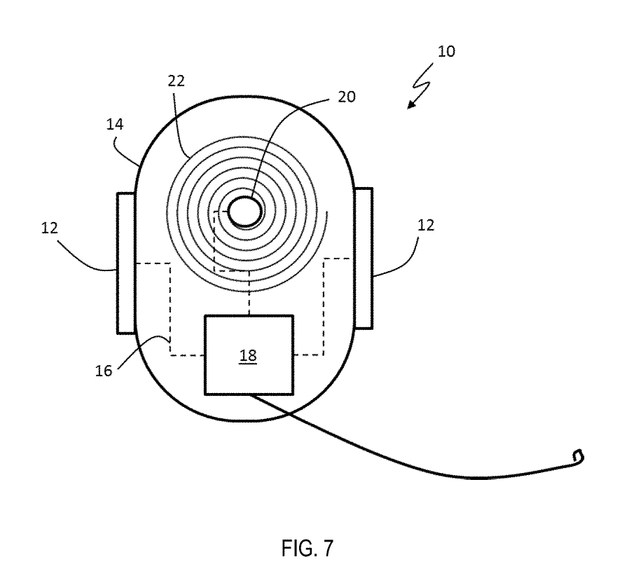

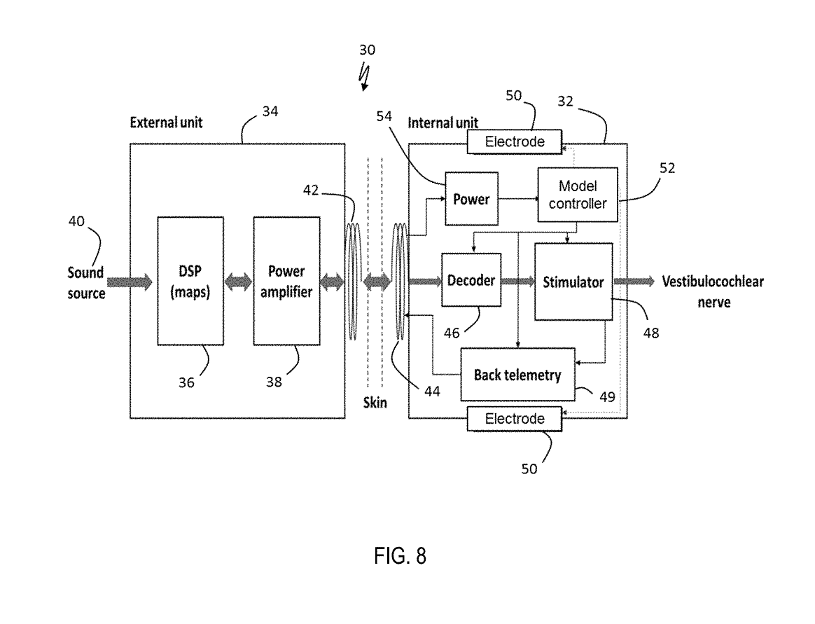

Referring to FIGS. 7 and 8, the present invention may comprise a cochlear implant 10 having integrated treatment electrodes 12 positioned on the outside surface of the housing 14 of the implant 10. Electrodes 12 are coupled via lines 16 to a receiver/stimulator 18 that can provide a voltage to electrodes 12, thereby producing a current field over the outer surface of housing 14. Receiver 18 is preferably coupled to a magnet 20 and associated coil 22 that can be inductively coupled to generate the DC voltage used to produce the appropriate electrical current across treatment electrodes 12. It should be recognized that conventional circuity for voltage regulation may be used. As explained above, electrodes 12 are designed to establish a predetermined DC treatment current field on the outside surface of housing 14 of cochlear implant 10 as a result of electric current flowing between electrodes 12, thereby eradicating any biofilm that has formed on shell 14 of cochlear implant 10.

As seen in FIG. 8, a cochlear implant system 30 may comprise an internal unit 32 that is implanted under the skin of a patient and an external unit 34 mounted on the outside of the skin of the patient. External unit 34 comprises a digital signal processor 36 and power amplifier 38 for receiving sounds from a sound source 40 and converting the sounds into a digital signal. External unit 34 transmits the digital signals representing the external sounds to internal unit 32. Internal unit 32 includes a decoder 46 that receives and transforms the digital signals representing the external sounds to a stimulator 48 that stimulates the vestibulocohlear nerve of the patient so that the sounds are heard by the patient. Back telemetry 49 may be provided for feedback to external unit 34. Antennas 42 and 44 may be used for the transmission of energy via wireless induction, for transmission of digital signals, or both.

Internal unit 32 is provided with a pair of electrodes 50 mounted on an exterior surface thereof as described above. A controller 52 is interconnected to the power source 54 of internal unit 32 and electrodes 46 as well as and decoder 46 and stimulator 48. Controller 52 is programmed to perform a DC treatment function as desired, e.g., in respond to an external signal received by decoder 46, or according to a predetermined schedule. Otherwise, controller 52 is programmed to allow internal unit 32 to function as a conventional cochlear implant. Controller 52 may also be configured to sample changes in the redox properties of electrodes 50. Any attached biofilm will change the redox properties, which can be characterized by electrochemical approaches such as cyclic volumetric method. Thus, the present invention could also serve as a biofilm sensor that monitors implant system 30 for any biofilm formation on the surface of implant system 30 and then provides for DC treatment if controller 52 determines that treatment is necessary. Power source 54 may be the battery for the cochlear implant circuity, a dedicated power source, or an inductive coil that can be magnetically coupled to produce a voltage for use by controller 52 to produce the direct current via the pair of electrodes 50.

Like cochlear devices, other medical implants are also known to have biofilm infections. For example, many bone fracture patients suffered from bacterial infection after surgery. The present invention can be applied to those implants. Because they do not carry charging units as the cochlear implant, implantable receiver coils will need to be added to wirelessly deliver DC. With this modification, it is possible to not only control bacterial biofilm formation, but also achieve electric stimulation of osteoblast without skin piecing.

### CONCLUSION

The present invention demonstrates that the two typical pathogenic bacteria associated with cochlear implants, P. aeruginosa and S. aureus, can be efficiently killed by low-level DC delivered with wireless coupling induction. The killing effect of low-level current was time dependent. This approach could avoid skin piercing, and can be applied to cochlear and other implants to monitor and eradicate biofilms on demand.

As described above, the present invention may be a system, a method, and/or a computer program associated therewith and is described herein with reference to flowcharts and block diagrams of methods and systems. The flowchart and block diagrams illustrate the architecture, functionality, and operation of possible implementations of systems, methods, and computer programs of the present invention. It should be understood that each block of the flowcharts and block diagrams can be implemented by computer readable program instructions in software, firmware, or dedicated analog or digital circuits. These computer readable program instructions may be implemented on the processor of a general purpose computer, a special purpose computer, or other programmable data processing apparatus to produce a machine that implements a part or all of any of the blocks in the flowcharts and block diagrams. Each block in the flowchart or block diagrams may represent a module, segment, or portion of instructions, which comprises one or more executable instructions for implementing the specified logical functions. It should also be noted that each block of the block diagrams and flowchart illustrations, or combinations of blocks in the block diagrams and flowcharts, can be implemented by special purpose hardware-based systems that perform the specified functions or acts or carry out combinations of special purpose hardware and computer instructions.

What is claimed is:

1. A medical implant, comprising:
a housing having an exterior surface and enclosing a power source; and
a pair of electrodes mounted to the exterior surface of the housing and interconnected to the power source to produce a direct current over the exterior surface of the housing.

2. The medical implant of claim 1, wherein the power source is a coil that may be inductively coupled to generate a voltage sufficient to produce the direct current over the exterior surface of the housing.

3. The medical implant of claim 2, wherein the direct current over the exterior surface of the housing has a density of between 0.1 and 100 μA/cm².

4. The medical implant of claim 3, wherein the medical implant is a cochlear implant.

5. The medical implant of claim 4, wherein the housing further includes a controller coupled to the power source and the pair of electrodes to control when the pair of electrodes produces the electrical current over the exterior surface of the housing.

6. The medical implant of claim 5, wherein the controller is programmed to produce the electrical current over the exterior surface of the housing for at least one hour.

7. The medical implant of claim 6, wherein the controller is programmed to sample the pair of electrodes to determine whether a biofilm is formed by monitoring the redox properties of the pair of electrodes.

8. The medical implant of claim 7, wherein the controller is programmed to produce the electrical current over the exterior surface of the housing in response to determining that the redox properties indicate the presence of a biofilm.

9. The medical implant of claim 1, wherein the power source is a battery positioned in the housing.

10. A method of treating for a biofilm, comprising the steps of:
providing a medical implant having a power source and a pair of electrodes position to produce a direct current over an exterior surface of the medical implant; and
producing the direct current over an exterior surface of the medical implant for a predetermined time and at a predetermined current density if a biofilm is present on the exterior surface of the medical implant.

11. The method of claim 10, wherein the power source includes an inductive coil.

12. The method of claim 12, wherein the step of producing the direct current over an exterior surface of the medical implant comprises the step of wirelessly inducing a voltage in the inductive coil of the power source.

13. The method of claim 12, wherein the medical implant is a cochlear implant.

14. The method of claim 13, wherein the predetermined current density is between 0.1 and 100 μA/cm².

15. The method of claim 14, wherein the predetermined time period is at least one hour.

16. The method of claim 15, further comprising the step of sampling the pair of electrodes to determine whether a biofilm is formed by monitoring the redox properties of the pair of electrodes.

17. The method of claim 16, wherein the step of producing the direct current over an exterior surface of the medical implant is performed if the step of sampling the pair of electrodes to determine whether a biofilm has altered the redox properties of the pair of electrodes determines that the biofilm is present.
